(19) United States
(12) Patent Application Publication
(10) Pub. No.: US 2022/0007957 A1
(43) Pub. Date: Jan. 13, 2022

(54) MEASUREMENT DEVICE, MEASUREMENT SYSTEM, AND DETERMINATION METHOD

(71) Applicant: TERUMO KABUSHIKI KAISHA, Tokyo (JP)

(72) Inventors: Shin Maki, Ebina-shi (JP); Tomoko UEMURA, Hadano-shi (JP)

(73) Assignee: TERUMO KABUSHIKI KAISHA, Tokyo (JP)

(21) Appl. No.: 17/449,138

(22) Filed: Sep. 28, 2021

Related U.S. Application Data
(63) Continuation of application No. PCT/JP2020/013130, filed on Mar. 24, 2020.

(30) Foreign Application Priority Data
Mar. 28, 2019 (JP) 2019-064855

(51) Int. Cl. A61B 5/0531; A61B 5/00

(52) U.S. Cl. CPC A61B 5/0531; A61B 2560/04; A61B 5/6804; A61B 5/0004

(57) ABSTRACT

A measurement device capable of executing measurement processing of impedance of a living body by applying a current to the living body includes three or more electrodes; and a control unit configured to control application of a current, in which the control unit measures the impedance of the living body when a current is applied to the living body for a predetermined plurality of combinations of electrodes constituted by a plurality of electrodes selected from the three or more electrodes, and determines one combination to be used for the measurement processing of impedance of the living body among the predetermined plurality of combinations based on the measured impedance.

### CROSS-REFERENCES TO RELATED APPLICATIONS

This application is a continuation of International Application No. PCT/JP2020/013130 filed on Mar. 24, 2020, and claims the benefit of Japanese Application No. 2019-064855 filed on Mar. 28, 2019, the entire content of each of which is incorporated herein by reference.

### FIELD OF THE INVENTION

The present disclosure relates to a measurement device, a measurement system, and a determination method.

### BACKGROUND DISCUSSION

Devices for measuring moisture contained in a living body by causing current to flow therethrough to measure its impedance are known in the related art. For example, JP-A-2001-218748 discloses a pulmonary water amount display device that measures and displays a moisture amount of a lung using such a bioelectrical impedance method.

In order to cause current to flow through the living body using such a device, it is known to connect, i.e., bring into contact, an electrode with the living body. However, a penetration depth of the current applied to the living body varies depending on a connection position of the electrode to the living body. Therefore, depending on the connection position of the electrode to the living body, the current may not reach a position where moisture is contained in the living body, and moisture contained in the living body may not be accurately measured. For example, when the penetration depth of the current applied to the living body is small, even if moisture is stored in the lung due to pulmonary edema or the like, it may not be possible to measure the impedance of a storage position of the moisture. In such a case, it is not possible to accurately diagnose a state of the living body. The connection position of the electrode where the penetration depth increases is different for each living body (i.e., individual).

An object of the present disclosure is to provide a measurement device, a measurement system, and a determination method that are capable of measuring moisture contained in a living body with higher accuracy.

### SUMMARY

A measurement device according to a first aspect of the present disclosure capable of executing impedance measurement processing of a living body by applying a current to the living body, the measurement device including: three or more electrodes; and a control unit configured to control application of a current, in which the control unit measures the impedance of the living body when a current is applied to the living body for a predetermined plurality of combinations of electrodes constituted by a plurality of electrodes selected from the three or more electrodes, and determines one combination to be used for the measurement processing of impedance of the living body among the predetermined plurality of combinations based on the measured impedance.

In the measurement device according to one embodiment of the present disclosure, the control unit applies a plurality of alternating currents having different frequencies to the living body, measures a resistive component and a capacitive component of the impedance of the living body, respectively plots the measured components on a horizontal axis and a vertical axis of two-dimensional coordinates, and determines one combination to be used for the measurement processing of impedance of the living body among the predetermined plurality of combinations using a value of a capacitive component in a predetermined resistive component as an index.

In the measurement device according to one embodiment of the present disclosure, the electrode is disposed on a mounting tool configured to be mounted on a user of the measurement device.

In the measurement device according to one embodiment of the present disclosure, the combination of the electrodes includes two electrodes selected from the three or more electrodes.

In the measurement device according to one embodiment of the present disclosure, five or more of the electrodes are provided, and the combination of the electrodes includes four electrodes selected from the five or more electrodes.

In the measurement device according to one embodiment of the present disclosure, the predetermined plurality of combinations are all combinations of electrodes arranged on a straight line among the five or more electrodes.

In the measurement device according to one embodiment of the present disclosure, the predetermined plurality of combinations are all combinations of electrodes in which the four electrodes are arranged in a rectangular shape among the five or more electrodes.

In the measurement device according to one embodiment of the present disclosure, the predetermined plurality of combinations are all combinations that the three or more electrodes are capable of taking.

A measurement system according to a second aspect of the present disclosure including a measurement device and an information processing device, in which the measurement device includes three or more electrodes, and a communication unit configured to transmit, to the information processing device, a result of measurement of impedance of a living body when a current is applied to the living body for a predetermined plurality of combinations of electrodes constituted by a plurality of electrodes selected from the three or more electrodes, and the information processing device includes a control unit configured to determine one combination to be used for measurement processing of the impedance of the living body among the predetermined plurality of combinations based on the result of measurement of the impedance of the living body.

A determination method according to a third aspect of the present disclosure executed by a measurement device that includes three or more electrodes and that is capable of executing measurement processing of impedance of a living body by applying a current to the living body, the determination method including: applying a current to the living body for a predetermined plurality of combinations of electrodes constituted by a plurality of electrodes selected from the three or more electrodes; measuring the impedance of the living body when a current is applied to the living body; and determining one combination to be used for the measurement processing of impedance of the living body among the predetermined plurality of combinations based on the measured impedance.

According to the present disclosure, it is possible to provide a measurement device, a measurement system, and a determination method that are capable of measuring moisture contained in a living body with higher accuracy.

### BRIEF DESCRIPTION OF THE DRAWINGS

FIG. 1 is a functional block diagram showing a schematic configuration of a measurement device according to a first embodiment;

FIG. 2 is a schematic view showing an example of a mounting tool in which electrodes constituting an electrode unit in FIG. 1 are arranged;

FIG. 3 is a schematic diagram showing a four-terminal method executed by the measurement device in FIG. 1;

FIG. 4 is a flowchart showing an example of processing executed by a control unit in FIG. 1;

FIG. 5 is a diagram showing a combination of electrodes arranged on a straight line in the arrangement of the electrodes in FIG. 2;

FIG. 6 is a diagram showing a combination of electrodes arranged in a rectangular shape in the arrangement of the electrodes in FIG. 2;

FIG. 7 is a schematic diagram showing a state in which a current flows through a living body;

FIG. 8 is a diagram schematically showing a flow of the current in an equivalent circuit of the living body;

FIG. 9 is a diagram schematically showing the flow of the current in the equivalent circuit of the living body;

FIG. 10 is a diagram schematically showing the flow of the current in the equivalent circuit of the living body;

FIG. 11 is a diagram showing an example of a Cole-Cole plot;

FIG. 12 is a functional block diagram showing a schematic configuration of a measurement system according to a second embodiment; and

FIG. 13 is a sequence diagram showing an example of processing performed by the measurement system in FIG. 12.

### DETAILED DESCRIPTION

Hereinafter, embodiments of a measurement device, a measurement system, and a determination method according to the present disclosure will be described with reference to drawings. In the drawings, common members are denoted by the same reference numerals.

### First Embodiment

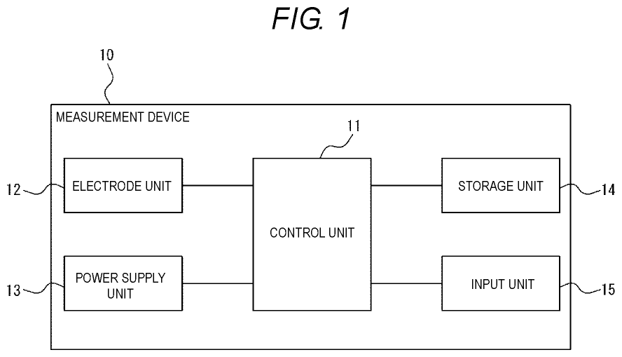

FIG. 1 is a functional block diagram showing a schematic configuration of a measurement device 10 according to a first embodiment. The measurement device 10 is a device capable of measuring impedance of a living body based on a bioelectrical impedance method. That is, the measurement device 10 can measure the impedance of a specific position of the living body by applying a current to the living body. By measuring the impedance, the measurement device 10 can estimate a moisture amount or record a change in the moisture amount at the specific position. For example, when there is a large amount of moisture at the specific position, electricity is more likely to flow due to an influence of moisture as compared with when there is a small amount of moisture. That is, a resistance at the specific position is reduced. By using this principle, the measurement device 10 can estimate the moisture amount or record the change in the moisture amount at the specific position.

In the present embodiment, it is assumed that the measurement device 10 measures the impedance of a right lung of the living body serving as the specific position. By measuring the impedance of the right lung, it is possible to estimate whether moisture is stored in the right lung. The specific position is not limited to the right lung. The specific position may be a left lung. The specific position may be a site other than the lung, such as a calf. The specific position may be any site to be inspected for a moisture storage state.

As shown in FIG. 1, the measurement device 10 includes a control unit 11, an electrode unit 12, a power supply unit 13, a storage unit 14, and an input unit 15.

The control unit 11 controls and manages the entire measurement device 10 including functional units of the measurement device 10. The control unit 11 includes at least one processor. The control unit 11 is a processor such as a central processing unit (CPU) that executes a program defining a control procedure or a dedicated processor specialized for processing of each function.

The control unit 11 controls an application of a current from the electrode unit 12 to the living body. The control unit 11 controls measurement processing of the impedance of the living body based on the bioelectrical impedance method. Before executing the measurement processing of the impedance of the living body, the control unit 11 determines a combination of electrodes to be used for the measurement processing of the impedance of the living body among the plurality of electrodes constituting the electrode unit 12. Details of combination determination processing executed by the control unit 11 will be described later.

The electrode unit 12 includes a plurality of electrodes. The number of electrodes provided in the electrode unit 12 may be appropriately determined according to a method of measuring the impedance performed by the measurement device 10 or the like. In the present embodiment, the control unit 11 measures the impedance using a combination of electrodes constituted by some of the electrodes constituting the electrode unit 12. Therefore, the electrode unit 12 includes electrodes whose number is larger than the number of the electrodes used in the measurement processing of the impedance. In the present embodiment, as will be described later, the impedance is measured based on a four-terminal method. Therefore, four terminals are used to measure the impedance. Therefore, in the present embodiment, the electrode unit 12 includes five or more electrodes.

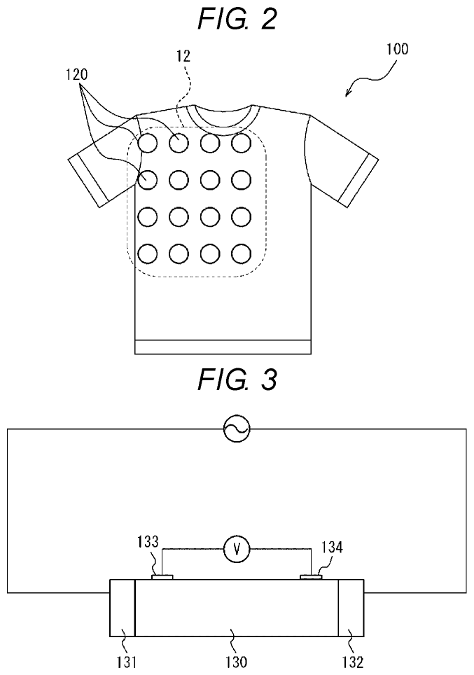

The electrodes constituting the electrode unit 12 are disposed on a mounting tool configured to be mounted on a user of the measurement device 10. FIG. 2 is a schematic view showing an example of a mounting tool in which electrodes constituting the electrode unit 12 are arranged. FIG. 2 shows an example in which the mounting tool is a T-shirt 100. In the example shown in FIG. 2, 16 electrodes 120 are disposed on the T-shirt 100. The 16 electrodes 120 are arranged at equal intervals in four vertical columns and four horizontal rows at positions where the electrodes 120 are in contact with the right chest of the user when the user wears the T-shirt 100. In the present embodiment, a description will be made below assuming that the electrode unit 12 includes 16 electrodes 120 and the electrodes 120 are arranged as shown in FIG. 2. With such an arrangement of the electrodes 120, when the user wears the T-shirt 100, the electrodes 120 come into contact with the right chest of the user, which is in the vicinity of the right lung of the user. Other functional units provided in the measurement device 10 are also attached to the T-shirt 100, which is the mounting tool.

The power supply unit 13 is a battery that supplies power to the functional units of the measurement device 10. The power supply unit 13 supplies power when, for example, a current is applied to the living body from the electrode unit 12.

The storage unit 14 can be a semiconductor memory, a magnetic memory, or the like. The storage unit 14 stores, for example, various types of information, a program for operating the measurement device 10, and the like. The storage unit 14 may also function as a work memory. For example, when the control unit 11 determines a combination of the electrodes by the combination determination processing of the electrodes, the storage unit 14 stores the determined combination of the electrodes. The determined combination of electrodes is, in other words, a combination of electrodes used in the measurement processing of the impedance of the living body.

The input unit 15 receives an operation input from a user, and includes, for example, operation buttons. The input unit 15 may be, for example, a touch screen, and may display an input region for receiving an operation input from the user on a part of a display device and receive a touch operation input by the user. The user can start the measurement of the impedance by the measurement device 10 by, for example, performing a predetermined operation input to the input unit 15.

The processing executed by the measurement device 10 according to the present embodiment will be further described. In the present embodiment, the measurement device 10 measures the impedance based on a method called a four-terminal method.

FIG. 3 is a schematic diagram showing the four-terminal method. In the four-terminal method, four terminals (electrodes) are connected to an impedance measurement target 130. Specifically, a first set of terminals including a first terminal 131 and a second terminal 132 are connected to both ends of the measurement target 130, and a current is applied to the measurement target 130 by the first set of terminals. Between the first set of terminals, a second set of terminals including a third terminal 133 and a fourth terminal 134 are connected to the measurement target 130, and a voltage between the second set of terminals is measured by the second set of terminals. Impedance between the second set of terminals can be calculated based on a current applied to the measurement target 130 by the first set of terminals and a voltage measured by the second set of terminals. Here, in the four-terminal method, since the current is negligibly small at a connection position of the third terminal 133 and the fourth terminal 134 with the measurement target 130, an electrode resistance of the third terminal 133 and the fourth terminal 134 can be ignored. Therefore, according to the four-terminal method, the impedance can be measured with high accuracy.

The measurement device 10 measures the impedance of the right lung using the four-terminal method. Here, when the impedance of the living body is measured, a penetration depth of the current applied to the living body varies depending on a connection position of the four electrodes to the living body. The connection position of the electrode where the penetration depth increases is different for each living body (i.e., individual).

In view of this, the measurement device 10 according to the present embodiment executes processing of determining a combination of the electrodes 120 used in the measurement processing of the impedance of the living body before executing the measurement processing of the impedance of the living body. Specifically, the measurement device 10 measures the impedance of the living body when a current is applied to the living body for a plurality of predetermined combinations of electrodes constituted by some of the electrodes 120, and determines one combination to be used for the measurement of the impedance of the living body based on the measured impedance.

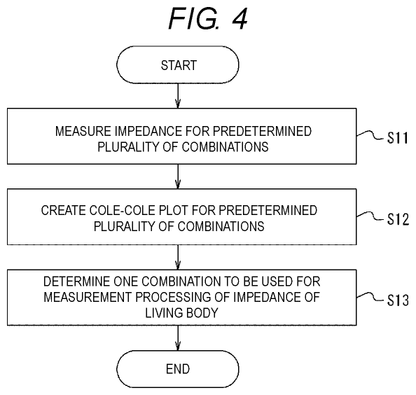

Here, details of the processing executed by the measurement device 10 will be described with reference to FIG. 4. FIG. 4 is a flowchart showing an example of processing executed by the control unit 11 of the measurement device 10, and is a flowchart relating to the processing of determining a combination of electrodes to be used for the measurement of the impedance of the living body.

The control unit 11 measures the impedance of the living body when the current is applied to the living body for the combination of the electrodes 120 constituted by some of the electrodes 120 provided in the electrode unit 12 (step S11). In the present embodiment, the control unit 11 measures the impedance of the living body when the current is applied to the living body for the combination of the electrodes 120 constituted by four electrodes in the 16 electrodes 120.

At this time, the control unit 11 measures the impedance of the living body when a current is applied to the living body for the plurality of predetermined combinations. For example, the predetermined plurality of combinations may be determined in advance, and may be stored in the storage unit 14.

For example, the predetermined plurality of combinations may be all combinations that can be taken when four electrodes 120 are selected from the 16 electrodes 120. In this case, the number of the predetermined plurality of combinations is 1820.

For example, the predetermined plurality of combinations may be predetermined specific combinations. That is, the predetermined plurality of combinations may be some of all the combinations that can be taken. The predetermined specific combination is stored in, for example, the storage unit 14.

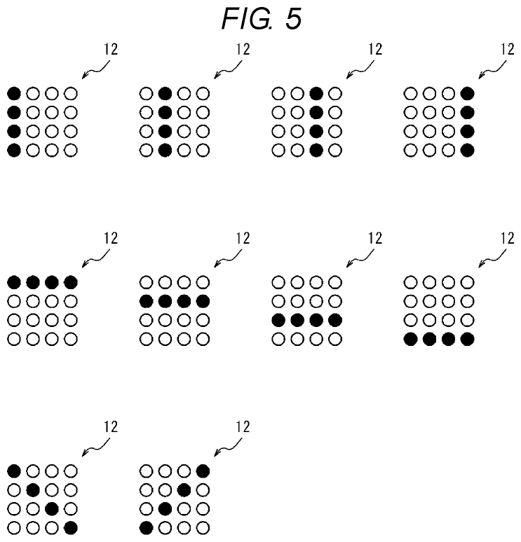

The specific combination may be, for example, all combinations of the electrodes 120 arranged on a straight line among the electrodes 120 provided in the electrode unit 12. For example, in the present embodiment, as shown in FIG. 2, the 16 electrodes 120 are arranged at equal intervals in four rows and four columns. Therefore, the number of combinations of the electrodes in which the four electrodes 120 are arranged on a straight line is ten in total, that is, a total of the number of combinations (four patterns) of the electrodes 120 arranged in a vertical direction, the number of combinations (four patterns) of the electrodes 120 arranged in a horizontal direction, and the number of combinations (two patterns) of the electrodes arranged in an oblique direction. FIG. 5 shows ten combinations of the electrodes 120 arranged on a straight line. In FIG. 5, filled points indicate the selected electrodes 120.

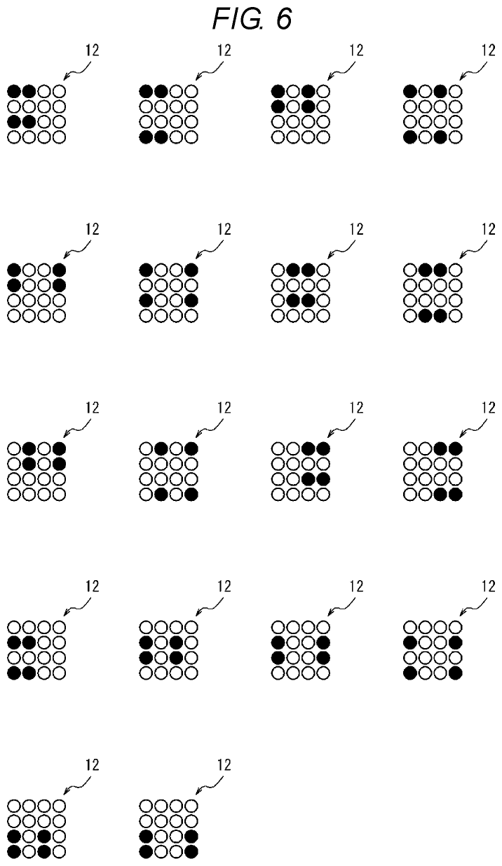

The specific combinations may be, for example, all combinations in which the arrangement of the four electrodes 120 among the electrodes 120 provided in the electrode unit 12 is in a rectangular shape. Here, the rectangular shape does not include a square shape. FIG. 6 shows combinations of the electrodes 120 arranged in the rectangular shape. In FIG. 6, filled points indicate the selected electrodes 120. The number of combinations in which the arrangement of the four electrodes 120 is in the rectangular shape is 18 as shown in FIG. 6.

The predetermined plurality of combinations are not limited to the example shown here, and may be any other plurality of combinations including four electrodes. The control unit 11 measures the impedance of all the predetermined plurality of combinations.

In the present embodiment, when measuring the impedance in step S11 in FIG. 4, the control unit 11 applies a plurality of alternating currents having different frequencies to the living body. The control unit 11 measures the impedance of the living body every time AC signals of different frequencies are applied. In the present embodiment, the control unit 11 measures a resistive component and a capacitive component of the impedance in step S11.

Next, the control unit 11 creates a Cole-Cole plot based on the impedance measured in step S11 (step S12). Specifically, the control unit 11 plots the resistive component and the capacitive component of the impedance measured in step S11 on the horizontal axis and the vertical axis of the two-dimensional coordinates, respectively. The control unit 11 creates a Cole-Cole plot by connecting the plots with a smooth line.

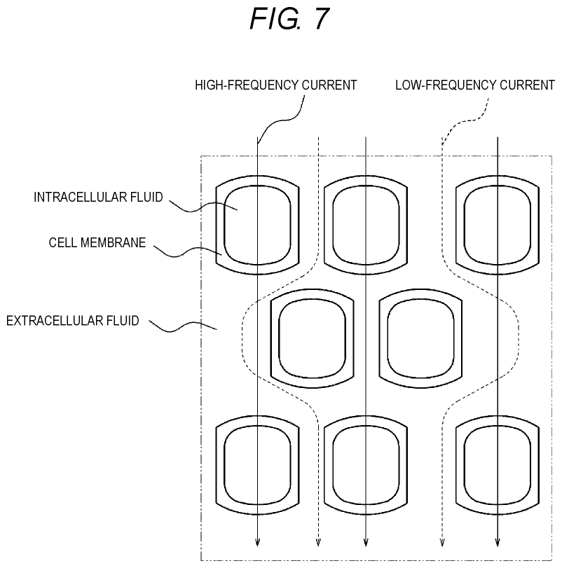

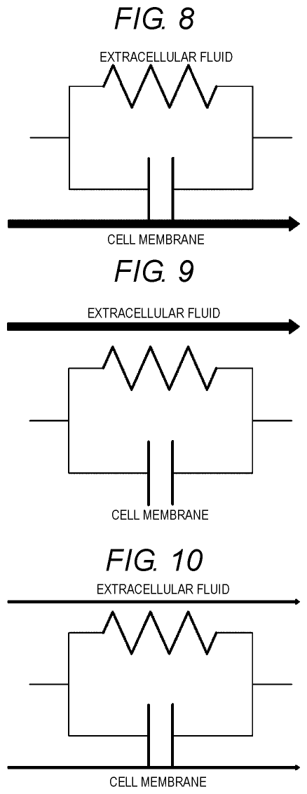

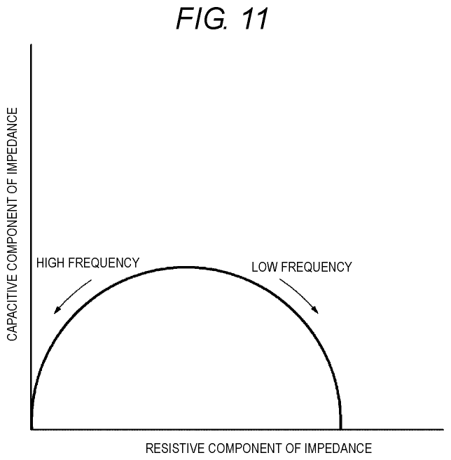

Here, the Cole-Cole plot will be described with reference to FIGS. 7 to 11. FIG. 7 is a schematic diagram showing a state in which a current flows through the living body. FIGS. 8 to 10 are diagrams schematically showing a flow of a current in an equivalent circuit of the living body. Specifically, FIG. 8 is a diagram showing a flow of a high-frequency current when the current is applied, FIG. 9 is a diagram showing a flow of a low-frequency current when the current is applied, and FIG. 10 is a diagram showing a flow of a current having an intermediate frequency between a high frequency and a low frequency when the current is applied. FIG. 11 is a diagram showing an example of the Cole-Cole plot.

In the living body, a cell can be considered as a system in which an electrolyte inside and outside the cell is insulated by a cell membrane having a high electrical insulation property. Due to the property, when a specific resistance is omitted, the living body can be expressed as an equivalent circuit in which resistance and capacitance are connected in parallel. In the equivalent circuit, as shown in FIGS. 8 to 10, it can be said that the resistance indicates a property of a extracellular fluid and the capacitance indicates a property of the cell membrane.

In the living body, the cell membrane has a property of having a larger capacitance than the extracellular fluid. The capacitance has a property of allowing high frequency waves to easily pass therethrough. Therefore, when the high-frequency current is applied to the living body, the high-frequency current flows so as to pass through the cell membrane as schematically shown by a solid line in FIG. 7. In this case, the resistance makes the current be less likely to flow than the capacitance. Therefore, as schematically indicated by arrows in FIG. 8, the current flows toward the capacitance. In this case, the resistance and the capacitance do not hinder the flow of the current. Therefore, as the frequency of the current is higher, the capacitive component of the impedance of the living body approaches 0, and the resistive component also approaches 0 except for a specific resistance which is not described.

On the other hand, the cell membrane has a property of having a larger resistance than the extracellular fluid. The capacitance has a property of making low frequency waves be less likely to pass therethrough. Therefore, when the low-frequency current is applied to the living body, the low-frequency current flows through the extracellular fluid between the cells due to the insulating property of the cell membrane, as schematically shown by a broken line in FIG. 7. In this case, when a change in the current is small and static capacitance of the capacitance is satisfied, the current does not flow through the capacitance, and the entire current flows toward the resistance. Accordingly, a current is less likely to flow in the capacitance than in the resistance. Therefore, as schematically indicated by arrows in FIG. 9, the current flows toward the resistance. In this case, the flow of the current is hindered by the resistance. Therefore, as the frequency of the current decreases, the capacitive component of the impedance of the living body approaches 0, and the resistive component increases.

When a current having an intermediate frequency between the high-frequency current and the low-frequency current is applied to the living body, the resistance and the capacitance have almost the same difficulty in the flow of the current. Therefore, as schematically indicated by arrows in FIG. 10, a current flows in both resistance and capacitance. Specifically, when the change in the current is small, the time during which the static capacitance of the capacitance is satisfied is long. While the static capacitance of the capacitance is satisfied, no current flows through the capacitance and a current flows through the resistance. Accordingly, the flow of the current is hindered by both the resistance and the capacitance. Therefore, in this case, the impedance of the living body has predetermined values in the capacitive component and the resistive component.

The control unit 11 applies a plurality of alternating currents having different frequencies to the living body, measures the resistive component and the capacitive component of the impedance of the living body, and respectively plots the resistive component and the capacitive component on the horizontal axis and the vertical axis of the two-dimensional coordinates. The control unit 11 creates a Cole-Cole plot as shown in FIG. 11 as an example by smoothly connecting a plurality of plots corresponding to a plurality of different frequencies by a line. As shown in FIG. 11, as the frequency of the applied current increases, the capacitive component of the impedance of the living body approaches 0, and the resistive component also approaches 0. As the frequency of the applied current decreases, the capacitive component of the impedance of the living body approaches 0, and the resistive component increases. Further, when a current having an intermediate frequency between the high-frequency current and the low-frequency current is applied, the capacitive component and the resistive component each have a predetermined value. Therefore, as shown in FIG. 11, the Cole-Cole plot has a semicircular shape in which the center is raised.

In step S12 in FIG. 4, the control unit 11 creates a Cole-Cole plot as shown in FIG. 11 for all of the predetermined plurality of combinations. After measuring the impedance for all the predetermined plurality of combinations in step S11, the control unit 11 may create the Cole-Cole plot for all the predetermined plurality of combinations in step S12, or may sequentially execute, for all the predetermined plurality of combinations, processing of measuring the impedance for one combination and creating the Cole-Cole plot for the one combination.

When creation of the Cole-Cole plots is completed for all the predetermined plurality of combinations in step S12, the control unit 11 determines one combination to be used for the measurement processing of the impedance of the living body among the combinations of the electrodes for which the Cole-Cole plots are created (step S13). At this time, the control unit 11 may determine one combination most suitable for the measurement processing of the impedance of the living body among the plurality of combinations of the electrodes for which the Cole-Cole plots are created as one combination to be used for the measurement processing of the impedance of the living body. The control unit 11 determines one combination most suitable for the measurement processing of the impedance of the living body as one combination to be used for the measurement processing of the impedance of the living body using, for example, a predetermined algorithm stored in the storage unit 14.

For example, the control unit 11 may determine one combination with reference to a specific index related to the Cole-Cole plot. Specifically, the control unit 11 may determine one combination using a value of a capacitive component in a predetermined resistance value component as an index. For example, the control unit 11 may determine one combination using a value of a capacitive component at a center of the Cole-Cole plot as the index. For example, the larger the penetration depth of the current applied to the living body, the greater the influence of the capacitance due to the cell membrane. That is, the capacitive component of the impedance increases. Therefore, the control unit 11 may determine, for example, a combination having the highest value of the capacitive component at the center of the Cole-Cole plot among the plurality of combinations as one combination to be used for the measurement processing of the impedance of the living body. The control unit 11 is not limited thereto, and may determine one combination most suitable for the measurement processing of the impedance of the living body as one combination to be used for the measurement processing of the impedance of the living body using any index capable of determining that the penetration depth of the current applied to the living body is large.

In this manner, the control unit 11 determines one combination to be used for the measurement processing of the impedance of the living body according to the flow in FIG. 4, and then executes the measurement processing of the impedance of the living body using the electrode 120 of the determined combination.

As described above, according to the measurement device 10 in the present embodiment, the control unit 11 measures the impedance of the living body when a current is applied to the living body for the predetermined plurality of combinations of electrodes constituted by a plurality of electrodes selected from the five or more electrodes 120, and determines one combination to be used for the measurement processing of the impedance of the living body among the predetermined plurality of combinations based on the measured impedance. Accordingly, the control unit 11 can execute the measurement processing of the impedance of the living body using the combination of the electrodes 120 in which the penetration depth of the current applied to the living body is larger. Therefore, according to the measurement device 10, the moisture contained in the living body can be measured with higher accuracy.

According to the measurement device 10 in the present embodiment, the electrode 120 is disposed on a mounting tool configured to be mounted on the user of the measurement device 10. Therefore, a positional relationship between the electrode 120 and the living body is less likely to change in a state in which the user is mounted with the mounting tool.

The measurement device 10 according to the present embodiment includes five or more electrodes 120, and the combination of the electrodes 120 includes four electrodes selected from the five or more electrodes 120. Accordingly, the measurement device 10 according to the present embodiment can be formed as a device that measures the impedance based on the four-terminal method.

In the measurement device 10 according to the present embodiment, the control unit 11 measures the impedance of the living body when a current is applied to the living body for the predetermined plurality of combinations. When the predetermined plurality of combinations are all the combinations that the electrode 120 can take, the control unit 11 can determine one combination to be used for measuring the impedance of the living body after verifying all combinations.

In the four-terminal method, it is known that accuracy of measuring the impedance is easily improved when the four electrodes 120 are arranged on a straight line or arranged in the rectangular shape. Therefore, when the predetermined plurality of combinations are, among the plurality of electrodes 120, all the combinations of the electrodes 120 arranged on the straight line and all the combinations in which the four electrodes 120 are arranged in the rectangular shape, it is easy to select one combination suitable for measuring the impedance of the living body while narrowing down the arrangement of the electrodes 120 for which the Cole-Cole plot is to be created to a specific arrangement.

In the above embodiment, the controller 11 may execute further processing. For example, in step S11 and step S12 in FIG. 4, the control unit 11 may measure the impedance for the predetermined plurality of combinations, create the Cole-Cole plot, and then determine whether at least one of the created Cole-Cole plots is included in a range of a predetermined threshold value. The predetermined threshold value is a threshold value indicating an allowable range for measuring the impedance of the living body, and is stored in advance in the storage unit 14, for example. When it is determined that at least one of the created Cole-Cole plots is included in the range of the predetermined threshold value, the control unit 11 determines one combination to be used for measuring the impedance of the living body from the combinations of the electrodes for which the Cole-Cole plots are created. When it is determined that none of the created Cole-Cole plots is included in the range of the predetermined threshold value, the control unit 11 can determine that there is no combination suitable for measuring the impedance of the living body among the combinations of the electrodes for which the Cole-Cole plots are created. In this case, the impedance may be measured for a predetermined plurality of other combinations different from the predetermined plurality of combinations of the electrodes for which the Cole-Cole plots are created, and the Cole-Cole plots may be created. Similarly, the control unit 11 determines whether at least one of the created Cole-Cole plots is included in the range of the predetermined threshold value for the predetermined plurality of other combinations. Accordingly, the control unit 11 can select one combination suitable for measuring the impedance of the living body from the combinations suitable for measuring the impedance of the living body.

When it is determined that there is no combination suitable for measuring the impedance of the living body for the predetermined plurality of other combinations described above, the control unit 11 further executes similar processing for a predetermined plurality of other combinations. The control unit 11 can repeat the above processing until it is determined that there is a combination suitable for measuring the impedance of the living body. As a result of repeating the above processing, when it is determined that there is no combination suitable for measuring the impedance of the living body for all the combinations that the electrodes 120 provided in the electrode unit 12 can take, the control unit 11 determines one combination most suitable for measuring the impedance of the living body from all the combinations that can be taken as the one combination to be used for the measurement processing of the impedance of the living body.

### Second Embodiment

In the first embodiment, an example has been described in which the present disclosure is implemented as the measurement device 10. However, the present disclosure does not necessarily need to be implemented as the measurement device 10. For example, the present disclosure may be implemented as a measurement system including a plurality of devices. An example in which the present disclosure is implemented as the measurement system will be described as a second embodiment.

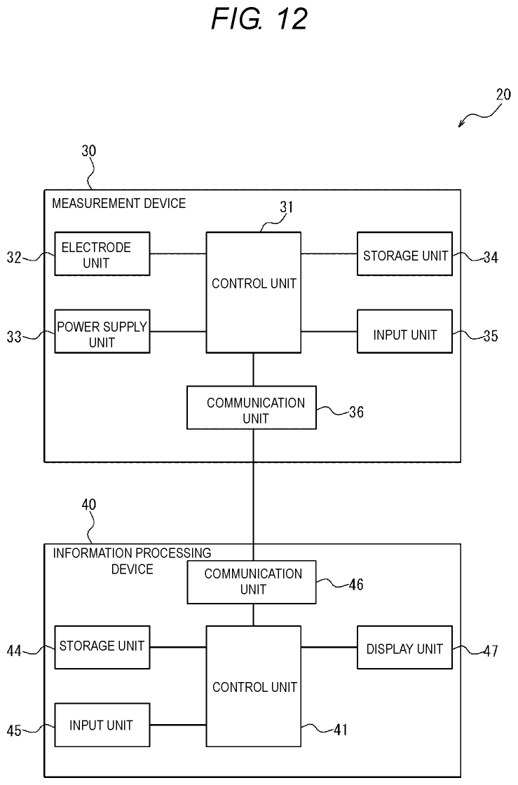

FIG. 12 is a functional block diagram showing a schematic configuration of a measurement system 20 according to the second embodiment. The measurement system 20 includes a measurement device 30 and an information processing device 40. The measurement device 30 and the information processing device 40 are connected to each other through wired communication or wireless communication so as to execute information communication with each other. The measurement system 20 achieves the function of the measurement device 10 according to the first embodiment by the measurement device 30 and the information processing device 40. Hereinafter, description of similar points as those according to the first embodiment will be appropriately omitted, and different points will be mainly described.

The measurement device 30 is the device capable of measuring impedance of a living body based on the bioelectrical impedance method. As shown in FIG. 12, the measurement device 30 includes a control unit 31, an electrode unit 32, a power supply unit 33, a storage unit 34, an input unit 35, and a communication unit 36.

In the measurement device 30 according to the present embodiment, configurations and functions of the electrode unit 32, the power supply unit 33, the storage unit 34, and the input unit 35 may be similar as the configurations and functions of the electrode unit 12, the power supply unit 13, the storage unit 14, and the input unit 15 of the measurement device 10 according to the first embodiment, and thus the description thereof will be omitted here.

In the measurement device 30, the control unit 31 controls and manages the entire measurement device 30 including functional units of the measurement device 30. In the present embodiment, the control unit 31 controls the application of a current from the electrode unit 32 to the living body based on a control signal received from the information processing device 40. The control unit 31 transmits information on the impedance of the living body measured using the electrode unit 32 to the information processing device 40 via the communication unit 36.

The communication unit 36 transmits and receives various types of information by executing wired communication or wireless communication with the information processing device 40. For example, the communication unit 36 receives, from the information processing device 40, a control signal that causes an application of the current to the living body. For example, the communication unit 36 transmits information on the impedance of the living body measured by the measuring device 30 using the electrode unit 32 to the information processing device 40.

The information processing device 40 is, for example, an electronic device such as a computer device or a terminal device. The information processing device 40 controls the application of current to the living body in the measurement device 30, and executes various types of information processing based on the information received from the measurement device 30. The information processing device 40 determines, for example, a combination of electrodes to be used for the measurement processing of the impedance of the living body among the plurality of electrodes constituting the electrode unit 32 of the measurement device 30. In the information processing device 40, for example, an application for executing processing of determining the combination of electrodes used for the measurement processing of the impedance of the living body may be installed in advance.

For example, as shown in FIG. 12, the information processing device 40 includes a control unit 41, a storage unit 44, an input unit 45, a communication unit 46, and a display unit 47.

The control unit 41 controls and manages the entire information processing device 40 including the functional units of the information processing device 40. The control unit 41 includes at least one processor. The control unit 41 is a processor such as the CPU that executes the program defining a control procedure or a dedicated processor specialized for processing of each function.

The control unit 41 generates a control signal for causing the measurement device 30 to apply a current to the living body, and transmits the control signal to the measurement device 30 via the communication unit 46. Before executing the measurement processing of the impedance of the living body by the measurement device 30, the control unit 41 determines a combination of electrodes to be used for the measurement processing of the impedance of the living body among the plurality of electrodes constituting the electrode unit 32 based on the information received from the measurement device 30. A method of determination may be similar as the method executed by the measurement device 10 according to the first embodiment.

The storage unit 44 can be a semiconductor memory, a magnetic memory, or the like. The storage unit 44 stores, for example, various types of information, a program for operating the information processing device 40, and the like. The storage unit 44 may also function as a work memory. For example, when the control unit 41 determines the combination of the electrodes by the determination processing of the combination of the electrodes, the storage unit 44 stores the determined combination of the electrodes.

The input unit 45 receives an operation input from the user, and includes, for example, operation buttons. The input unit 45 is, for example, a touch screen, and may display an input region for receiving an operation input from the user on a part of the display device and receive a touch operation input by the user. The user can start the control by the information processing device 40 by, for example, executing a predetermined operation input to the input unit 45 and thereby start the measurement of the impedance by the measurement device 30.

The communication unit 46 transmits and receives various types of information by executing wired communication or wireless communication with the measurement device 30. For example, the communication unit 46 transmits the control signal for causing the measurement device 30 to apply the current to the living body. For example, the communication unit 46 receives, from the measurement device 30, information on the impedance of the living body measured by the measurement device 30 using the electrode unit 32.

The display unit 47 is a display device which is a well-known display such as a light emitting diode (LED) display, a liquid crystal display (LCD), or an organic electroluminescence display (OELD). The display unit 47 displays various types of information. For example, the display unit 47 displays that the measurement processing of the impedance of the living body is being executed. Accordingly, the user who views the display can know that the measurement processing of the impedance of the living body is being executed.

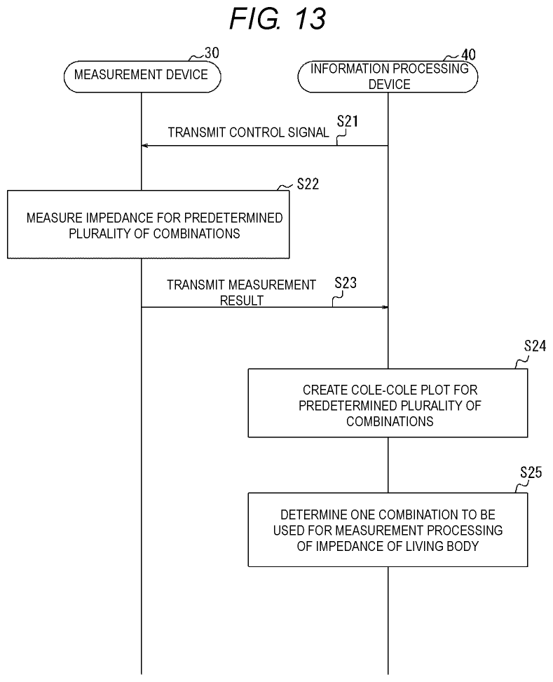

Here, details of the processing executed by the measurement system 20 will be described with reference to FIG. 13. FIG. 13 is a sequence diagram showing an example of processing performed by the measurement system 20 in FIG. 12, and is a sequence diagram related to processing of determining a combination of electrodes to be used for measuring the impedance of the living body.

In the processing of determining the combination of the electrodes to be used for measuring the impedance of the living body, first, the information processing device 40 transmits, to the measurement device 30, a control signal for executing the measurement of the impedance for a predetermined plurality of combinations of the electrodes 120 (step S21).

When a control signal is received from the information processing device 40, the measurement device 30 measures the impedance of the living body when the current is applied to the living body for the combination of the electrodes 120 constituted by some of the electrodes 120 provided in the electrode unit 12 (step S22). The specific processing in step S22 may be similar as that in step S11 in FIG. 4.

Next, the measurement device 30 transmits a result of the measurement of the impedance in step S22 to the information processing device 40 (step S23).

When the information processing device 40 receives the result of the measurement of the impedance executed by the measurement device 30, the information processing device 40 creates a Cole-Cole plot based on the impedance serving as the result of the measurement (step S24). The specific processing in step S24 may be similar as that in step S12 in FIG. 4.

When creation of the Cole-Cole plots is completed for all the predetermined plurality of combinations in step S24, the information processing device 40 determines one combination to be used for the measurement processing of the impedance of the living body among the combinations of the electrodes for which the Cole-Cole plots are created (step S25). The specific processing in step S25 may be similar as that in step S25 in FIG. 4. In this manner, the information processing device 40 determines one combination to be used for the measurement processing of the impedance of the living body, and then executes the measurement processing of the impedance of the living body using the electrodes 120 of the determined combination.

As described above, according to the measurement system 20 in the present embodiment, the measurement processing of the impedance of the living body can be executed using the combination of the electrodes 120 in which the penetration depth of the current applied to the living body is larger. Therefore, according to the measurement system 20, the moisture contained in the living body can be measured with higher accuracy.

In the above embodiment, a case has been described in which the impedance is measured based on the four-terminal method. However, the present disclosure is also applicable to a case in which the impedance is measured based on a method other than the four-terminal method, for example, a two-terminal method.

When the impedance is measured based on the two-terminal method, two terminals are used for the measurement of the impedance. Therefore, in this case, the electrode unit 12 may include three or more electrodes. In this case, the combination of the electrodes to be used for the measurement processing of the impedance of the living body includes two electrodes selected from three or more electrodes. In the case of the two-terminal method, the impedance can be measured with a smaller number of terminals.

In the above embodiment, it has been described that the electrode unit 12 is constituted by the 16 electrodes 120. However, the number of the electrodes 120 provided in the electrode unit 12 is not limited thereto. The electrode unit 12 may include an appropriate number of electrodes 120 according to a specification of the measurement device 10 or the like. As the number of the electrodes 120 provided in the electrode unit 12 increases, the number of candidates for the combination of the terminals used in the measurement processing of the impedance of the living body increases. Therefore, as the number of the electrodes 120 provided in the electrode unit 12 increases, the chance of occurrence of a combination in which the penetration depth of the current is larger is higher.

In the above embodiment, it has been described that the 16 electrodes 120 are arranged at equal intervals in four rows and four columns. However, the arrangement of the electrodes 120 is not limited thereto. The electrode 120 may be appropriately disposed in the mounting tool.

In the above embodiment, it has been described that the mounting tool is the T-shirt 100 and the impedance of the right lung of the living body is measured. However, the present disclosure is not limited to this aspect. As the mounting tool, an appropriate one may be used according to a position where the impedance is measured in the living body. For example, when the impedance of the calf of the living body is measured, the mounting tool may be implemented as a mounting tool to be mounted on the lower body, such as trousers or tights.

It is preferable that the mounting tool is formed of a material and in a form that are easily brought into close contact with the living body. Since the mounting tool is formed of a material and in a form that are easily brought into close contact with the living body, the electrode 120 disposed on the mounting tool is easily brought into contact with the living body.

The measurement device, the measurement system, and the determination method according to the present disclosure are not limited to the configurations specified in the embodiments described above, and various modifications can be made without departing from the gist of the invention described in the claims. For example, functions and the like in the components, the steps, and the like can be rearranged in a manner of not being logically contradictory, and a plurality of components, steps, and the like can be combined into one or divided.

### INDUSTRIAL APPLICABILITY

The present disclosure relates to a measurement device, a measurement system, and a determination method. The measurement device, the measurement system, and the determination method according to the present disclosure can be applied to, for example, a patient of pulmonary edema. According to the measurement device, the measurement system, and the determination method in the present disclosure, it is possible to measure the storage state of moisture in the patient of pulmonary edema with higher accuracy.

What is claimed is:

1. A measurement device capable of executing measurement processing of impedance of a living body by applying a current to the living body, the measurement device comprising:
three or more electrodes; and
a control unit configured to control application of a current, wherein
the control unit is configured to:
measure the impedance of the living body when a current is applied to the living body for a predetermined plurality of combinations of electrodes constituted by a plurality of electrodes selected from the three or more electrodes, and
determine one combination to be used for the measurement processing of impedance of the living body among the predetermined plurality of combinations based on the measured impedance.

2. The measurement device according to claim 1, wherein
the control unit applies a plurality of alternating currents having different frequencies to the living body, measures a resistive component and a capacitive component of the impedance of the living body, respectively plots the measured components on a horizontal axis and a vertical axis of two-dimensional coordinates, and determines one combination to be used for the measurement processing of impedance of the living body among the predetermined plurality of combinations using a value of a capacitive component in a predetermined resistive component as an index.

3. The measurement device according to claim 1, wherein
the electrode is disposed on a mounting tool configured to be mounted on a user of the measurement device.

4. The measurement device according to claim 1, wherein
the combination of the electrodes includes two electrodes selected from the three or more electrodes.

5. The measurement device according to claim 1, wherein
five or more of the electrodes are provided, and
the combination of the electrodes includes four electrodes selected from the five or more electrodes.

6. The measurement device according to claim 5, wherein
the predetermined plurality of combinations are all combinations of electrodes arranged on a straight line among the five or more electrodes.

7. The measurement device according to claim 5, wherein
the predetermined plurality of combinations are all combinations of electrodes in which the four electrodes are arranged in a rectangular shape among the five or more electrodes.

8. The measurement device according to claim 1, wherein
the predetermined plurality of combinations are all combinations that the three or more electrodes are capable of taking.

9. A measurement system including a measurement device and an information processing device, wherein
the measurement device includes
three or more electrodes, and
a communication unit configured to transmit, to the information processing device, a result of measurement of impedance of a living body when a current is applied to the living body for a predetermined plurality of combinations of electrodes constituted by a plurality of electrodes selected from the three or more electrodes, and
the information processing device includes a control unit configured to determine one combination to be used for measurement processing of the impedance of the living body among the predetermined plurality of combinations based on the result of measurement of the impedance of the living body.

10. The measurement system according to claim 9, wherein
the electrode is disposed on a mounting tool configured to be mounted on a user of the measurement device.

11. The measurement system according to claim 9, wherein
the combination of the electrodes includes two electrodes selected from the three or more electrodes.

12. The measurement system according to claim 9, wherein
five or more of the electrodes are provided, and
the combination of the electrodes includes four electrodes selected from the five or more electrodes.

13. A determination method executed by a measurement device that includes three or more electrodes and that is capable of executing measurement processing of impedance of a living body by applying a current to the living body, the determination method comprising:
applying a current to the living body for a predetermined plurality of combinations of electrodes constituted by a plurality of electrodes selected from the three or more electrodes;
measuring the impedance of the living body when a current is applied to the living body; and
determining one combination to be used for the measurement processing of impedance of the living body among the predetermined plurality of combinations based on the measured impedance.

14. The determination method according to claim 13, further comprising:
applying a plurality of alternating currents having different frequencies to the living body;
measuring a resistive component and a capacitive component of the impedance of the living body;
respectively plotting the measured components on a horizontal axis and a vertical axis of two-dimensional coordinates; and
determining one combination to be used for the measurement processing of impedance of the living body among the predetermined plurality of combinations using a value of a capacitive component in a predetermined resistive component as an index.
